(19) United States
(12) Patent Application Publication
(10) Pub. No.: US 2012/0277543 A1
(43) Pub. Date: Nov. 1, 2012

(54) SYSTEM AND METHOD FOR UPLOADING AND SECURING HEALTH CARE DATA FROM PATIENTS AND MEDICAL DEVICES TO TRUSTED HEALTH-USER COMMUNITIES

(75) Inventors: Joydip Homchowdhury, Round Rock, TX (US); Kimberlie Louise Cerrone, San Francisco, CA (US); Ratan Dev Bhardwaj, Phoenix, AZ (US)

(73) Assignee: TIATROS INC., San Francisco, CA (US)

(21) Appl. No.: 13/449,972

(22) Filed: Apr. 18, 2012

Related U.S. Application Data
(63) Continuation-in-part of application No. 13/096,887, filed on Apr. 28, 2011.

(51) Int. Cl. A61B 5/00

(52) U.S. Cl. 600/300

(57) ABSTRACT

Systems and methods of uploading patient medical and/or physiological data to the patient's associated CarePod are disclosed. Devices that are capable of sensing patient medical and/or physiological data are disclosed that are in authenticated and secure communication with the patient's CarePod. Doctors and other caregivers may be able to analyze the patient medical and/or physiological data in near real-time basis. Other devices, systems and methods are disclosed to provide authenticated and secure therapeutic treatment in possibly a closed loop system. Other devices, systems and methods are disclosed to guard against the inadvertent misdirection of patient medical and/or physiological data between medical devices and the patient's CarePod.

### CROSS-REFERENCE TO RELATED APPLICATIONS

This Patent Application is a Continuation-in-Part (CIP) Application, and claims the benefit of, a co-pending Application with a Ser. No. 13/096,887 filed by common Inventors of this Application on Apr. 28, 2011. The disclosure made in the application Ser. No. 13/096,887 is hereby incorporated by reference in its entirety.

### BACKGROUND

Changes in the nature and practice of medical care have occurred for numerous reasons—with ballooning costs and the efforts to contain the same being one among them. One way to improve the accuracy of health care, while reducing the cost, is to have the patient provide self-testing in the home environment as possible. For example, patients with high blood pressure (HBP) may have a BP monitor at home and have substantial knowledge about how to make accurate readings—which, over time, will give the patient's physician substantial information on the course of treatment. The same holds true for diabetes—patients have home glucose testing machines and the readings taken from them give the physician information on the course of treatment. In a similar way, some medical devices—some implantable (e.g. pacemakers) and others not—generate medical data about the patient in the course of their use. Additionally, biosensors (i.e., biological sensing elements) may be connected to a transducer to convert an observed response into a measurable signal—and generate relevant medical data as well.

### SUMMARY OF THE INVENTION

Systems and methods of uploading patient medical and/or physiological data to the patient's associated CarePod are disclosed. Devices that are capable of sensing patient medical and/or physiological data are disclosed that are in authenticated and secure communication with the patient's CarePod. Doctors and other caregivers may be able to analyze the patient medical and/or physiological data in near real-time basis. In addition, the present system may be able to perform the analysis in substantially real-time and present it to relevant caregivers—in order for the caregiver to render timely treatment or inform their decision-making process. Other devices, systems and methods are disclosed to provide authenticated and secure therapeutic treatment in possibly a closed loop system. Other devices, systems and methods are disclosed to guard against the inadvertent misdirection of patient medical and/or physiological data between medical devices and the patient's CarePod.

Other features and advantages of the present system are presented below in the Detailed Description when read in connection with the drawings presented within this application.

### BRIEF DESCRIPTION OF THE DRAWINGS

FIG. 1 shows a high level block diagram of a possible set of trusted users and possible modules for a system built in accordance with the principles of the present invention, and more particularly for medical applications built upon the system.

FIG. 2 depicts the concept of a social pod as made in accordance with several of the present embodiments.

FIG. 3 shows a flow chart of one embodiment of creating and authenticating a social pod within the context of a medical application.

FIG. 4 is a high level block diagram of a system architecture built according to the principles of the present application.

FIG. 5 shows a flow chart of one embodiment of a multimedia content engine as made in accordance with several of the present system embodiments.

FIG. 6 is one embodiment of a present system as built and hosted using existing network infrastructure.

FIGS. 7A and 7B show one embodiment of a present system as built for the treatment of PTSD for returning military veterans.

FIGS. 8A and 8B show one embodiment of a de-identifier module to de-link information within communications of the social pod that contains certain data that might identify patients receiving treatment.

FIG. 9 depicts one embodiment of a screen shot showing the functionality of a treatment plan set up for a patient by a physician.

FIG. 10 shows one embodiment of a program funding module that enables administrators of a social pod to raise funds for programs via the present system.

FIG. 11 depicts conventional methods where patient medical and/or physiological data is send from a medical device to the patient's caregivers.

FIG. 12 is one embodiment of a system and/or method of enabling secure and authenticated communications of patient medical data from a device to the patient's CarePod.

FIG. 13 is one embodiment of a system and/or method to prevent the accidental misdirection of patient medical data from a device to the patient's CarePod.

FIG. 14 is one embodiment of a system and/or method of providing secure and authenticated therapeutic treatment to a patient under guidance from doctors and other caregivers from the patient's CarePod.

### DETAILED DESCRIPTION

### Trusted Communities and/or Social Pods

In one possible aspect, the present embodiment may require that the physician communicate with a patient who is authenticated at the time of communication to the patient. In addition, the system stores and/or otherwise archives the interaction between the physician and the patient to form a part of the latter's EHR.

In another possible aspect, the present system may define a set of “trusted” users of the network. Such trusted users may need to be authenticated to establish their level of engagement and interaction with the system. Such authentication may be accomplished by any known method, manner or system for such authentication. Examples include password protection, challenge-response interactions, biometrics or the like.

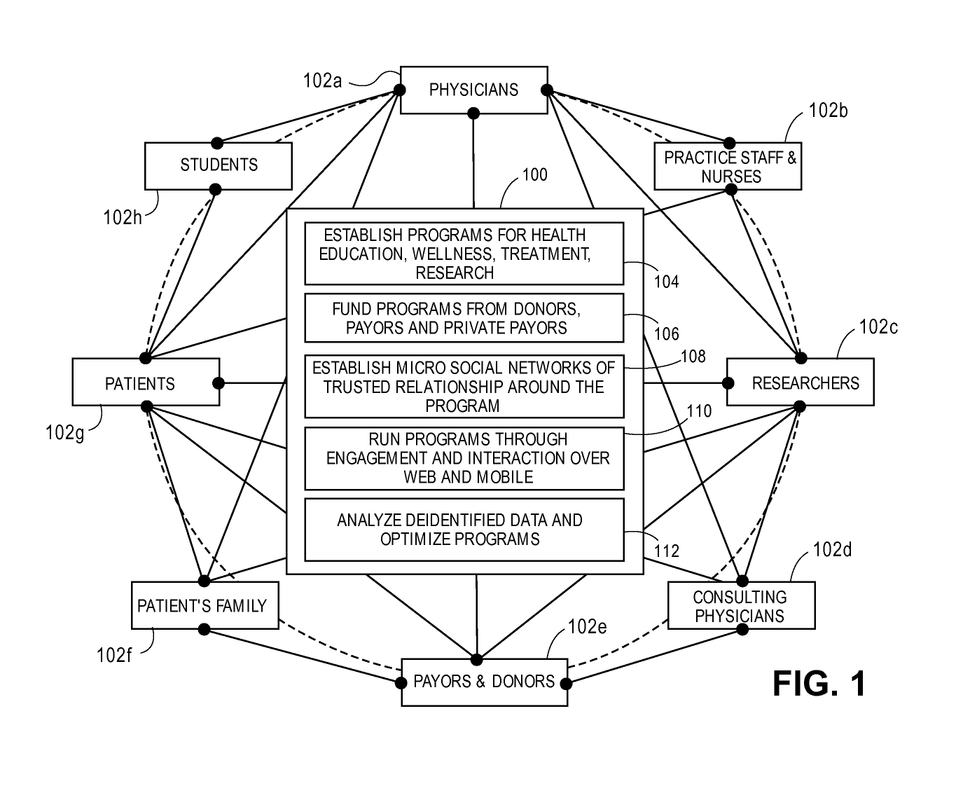

FIG. 1 describes a set of entities that might comprise a prototypical environment of trusted users. Users (collectively labeled 102) are shown interconnectedly with the present system 100 and, possibly, connected amongst themselves apart from system 100. A set of users might comprise the following types of individuals: physicians 102a, practice staff and nurses 102b, researchers 102c, consulting physicians 102d, payor and donors 102e, patient's friends and family members 102f, patients 102g and students 102h.

Each of the users 102 represent entities that may have known communication and computing devices (not shown) in order to affect a networked environment. For example, users 102 may variously have smart phones, cell phones, computers, tablets and the like that may be configured to run a secure, encrypted software environment, as might be presented in a browser or in any other known interfaces. It will be appreciated that the present system encompasses the use of all known devices and means of networked communication that would facilitate the present system as described herein.

The present system may also allow for easy dynamic management of the social pod. For example, the present system may allow for the addition and/or deletion of members in a seamless manner. To appreciate the flexibility of communities that the present system could enable, trusted communities might comprise one, two, or any number of members depending on their specific purpose. For mere exemplary purposes, communities may consist of:

a single member using a self-directed therapeutic intervention

doctor+doctor

doctor to pharmacy

doctor to health insurance agent, e.g., for utilization review

doctor+patient

doctor+patient+family

doctor+entire care team

patient+entire care team

doctor+multiple patients or multiple families

research team

research team+participants

wellness program enrollees

medical-educational program enrollees

The identity of every participant in a community may be authenticated using one or more conventional identity authentication methods each time the member signs on to the community or accesses a content file. The present system may incorporate a variety of conventional authentication methods; the specific method(s) used to authenticate the members of a given community may vary as appropriate to its specific purpose.

Communities may be moderated, or self-directed. One or more moderators may oversee some types of programs, being able, for example, to add new members, remove objectionable content, and update content files. Other types of programs may be completely unsupervised and self-directed.

Because the present system may ensure HIPAA privacy and security compliance, communications and medical records that contain personal health information may be shared among members of the community, synchronously and asynchronously, online and on tablets and mobile phones.

In addition to setting up and populating trusted communities, the present system may use a number of technical strategies to pre-set and enforce access rights to ensure the privacy of communications, and appropriately limit access to certain files. Easy-to-use and redundant methods assure that the moderator(s) exercise complete and dynamic control over which communications and medical records, or parts thereof, are available to everyone, and which are available only to a certain subset of the community.

System 100 may comprise a set of networked computers and/or processors—in communications possibly with computers, processors or mobile devices that are in the possession or under the control of the users 102. There are many desirable and optional features that system 100 provides to users 102 and to the various HCP that are connected to the users.

For example, system 100 may provide the following:

(1) establish networked infrastructure for programs for health, education, prevention, wellness, treatment and/or research (104);

(2) enable automated and/or distributed funding of programs from donors, granting organizations, payors and private payors (106);

(3) establish micro social networks of trusted relationships around the program;

(4) run programs through engagement and interactions over networks (e.g. intranets, the internet or the like) and mobile devices; and

(5) analyze de-identified data that flows through system 100 and optimize programs that are made in accordance with the present system (112).

One embodiment of analysis and optimization of the present system provides that the interactions of involving users and the present system provides a feedback mechanism to sharpen and improve the effectiveness of the system for treating or servicing its users. For example, one embodiment of the present system might be a Clinical Care and Education program that allows providers several means to capture the data about the effectiveness of their programs. The “social” interactions inherent in the solution may be captured by the system, for example as unstructured data. The built Query-Response service allows the system to get explicit feedback in a secure fashion. In addition, the Therapeutics module might allow the system to capture responses from their patients and participants e.g. level of pain, mood, etc., along with compliance data such as “Did you take all three dosages of the medicine, on time” etc. This data set allows the system and its designers (which could be the clinicians and researchers of the program itself) to look for correlation among a particular protocol and its effectiveness and make changes to their programs, be it therapeutics or course material, style of presentation, etc.

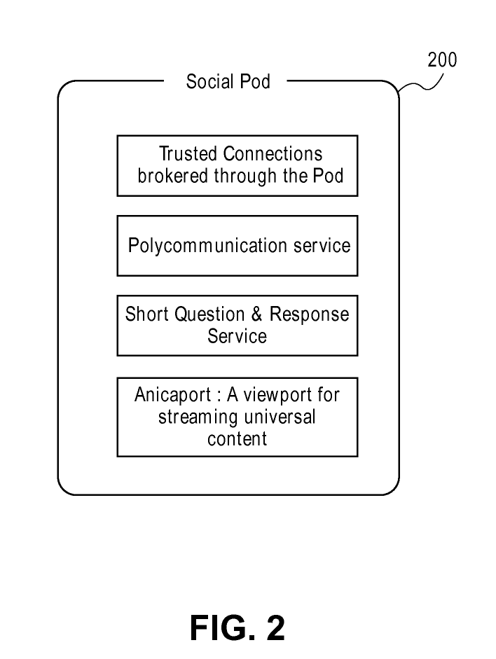

System 100 may be employed to create a networked “microcommunity” of users—a construct called a “social pod”. FIG. 2 depicts a social pod 200. Social pod 200 is enabled or otherwise hosted by system 100 as a set of interconnected computers, processors, mobile devices or the like. Desirable features of social pod 200 may include: a set of trusted connections brokered through the system; a polycommunication service (e.g. email, SMS, voicemail or the like); short question and response service; and a viewport and/or an application (called an “anicaport” for purposes of this application, as described below). This anicaport may act, at a high level, as a viewport for downloading, uploading, and/or streaming of content. Such content may be placed into appropriate formatting and made available to all or a subset of trusted users, possibly in some universal format. In one embodiment, a social pod may provide a restricted and secure way for a micro community of people organized around a specific outcome (e.g. clinical research, treatment of a medical condition, education for wellness etc.) to interact, collaborate, capture structured data, etc.

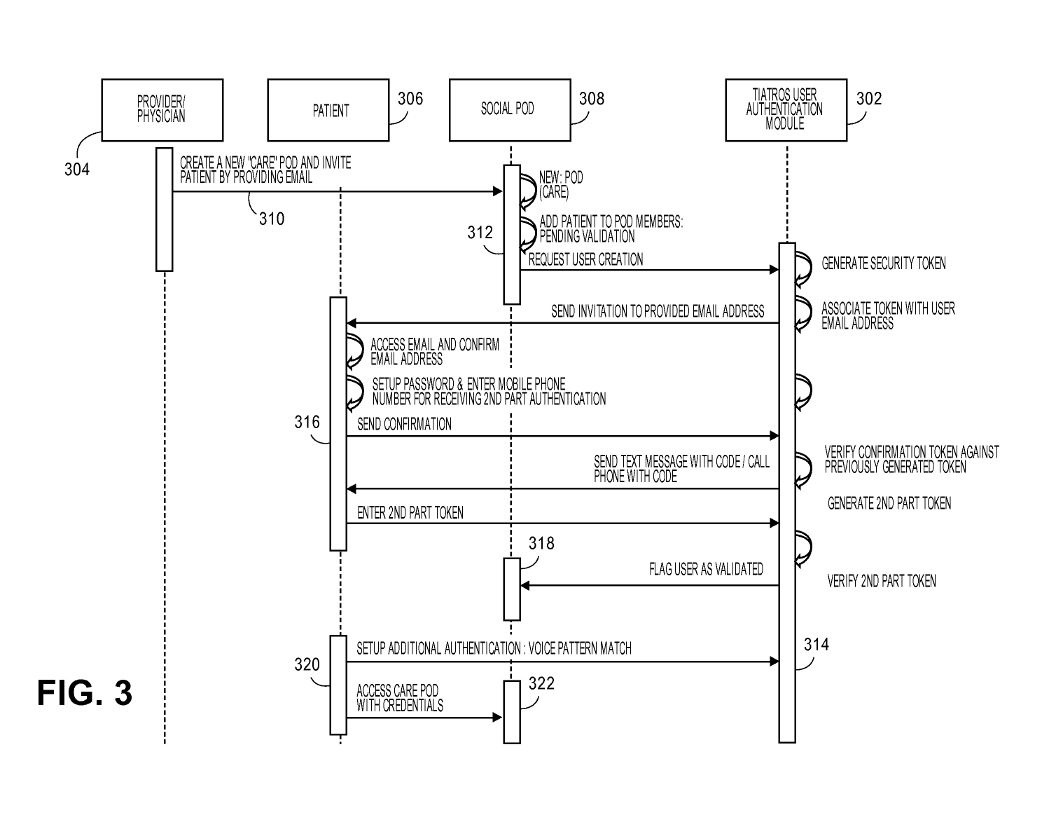

FIG. 3 depicts one embodiment of a method of creating a social pod. In this embodiment, the system may allow for a multi-part authentication procedure and mechanism. It will be appreciated, however, other mechanisms—with varying levels of authentication—may be set up and managed. It will be appreciated that the following description is merely by way of example and that other mechanisms and methods may be employed to created trusted communities and/or social pods.

Social pod 308 may be created by a provider, a physician or researcher 304 via the present system. Provider 304 alerts the system that a new “Care” pod is to be created and provider 304 may populate the pod by listing individuals (e.g. patient 306) and have the system invite patient 306 via some identified means of communication (e.g. by providing the patient's email address to the system) at 310. The system may manage social pod 308 as a set of data structures and/or routines to affect its creation and dynamic management. At 312, the system (via social pod 308 or the like) creates the new “Care” pod and adds patient 306 as a pod member, pending authentication. Pod 308 may then request the system to create patient as a User—in this example, via a request to the system's authentication module 302.

Authentication module 302 may perform such actions as shown at 314. To wit, module 302 may generate a security token and associate the token with the user's email address or any other identifier. Module 302 may return an invitation to the identified email address of the putative new user/patient 306. Patient 306 may then (at 316) access her email and confirm the address, setup a user password and enter other means of communication for the system (such as mobile phone number or the like). This other means of communication may be used to receive a second part authentication for the user. Once initial confirmation is received from patient 306, module 302 may confirm the token against the previously generated token (at 314) and send a text message to the mobile phone (or call the phone directly) with a second part token. Patient 306 may enter the second part token and return to module 302 for further authentication. If module 302 confirms the second part token, module 302 may signal to pod 308 that there is a trusted individual/user at 318.

Additional authentication means may optionally be set up, as desired. For example patient 306 may set up a voice recognition match for further authentication at 320, back to module 302. As time goes forward, patient 308 is then considered a trusted user and may access the pod with suitable credentials at 322.

In one embodiment, the present system may provide flexibility in setting up trusted relationships. For this, it may be desirable to establish that the forms of identifications provided by the user are indeed accessible by the user. For this, the present system may establish such multi-part authentication mechanism as desired. In addition, the administrators or providers of the system can choose the levels of authentication required for trusted users, with a basic minimum possibly designed.

### System Architecture

Having described one aspect of the present system—i.e. the notion of trusted users and the social pod, one or more suitable architecture embodiments for the construction of the present system will now be described. In addition, it will be shown how one embodiment of the present system may leverage existing internet and other infrastructures for efficient build-out of the present system.

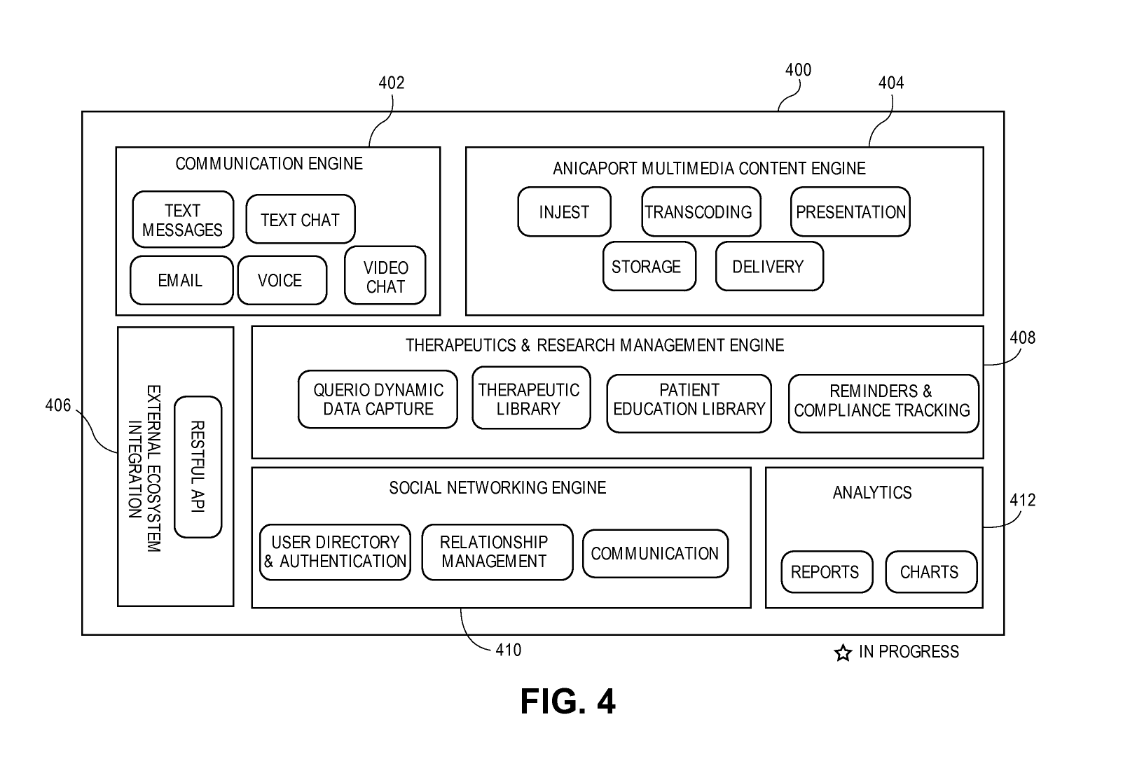

FIG. 4 depicts one embodiment of an architecture of a system that may perform in accordance with the teachings of the present invention. System 400 may advantageously comprise multiple modules for the creation and dynamic operation of the present system. Such modules may comprise the following: communication engine 402, multimedia content engine 404, external ecosystem integration module 406, therapeutic and research management engine 408, social networking engine 410 and analytic engine 412. Each module/engine will be discussed in turn below.

Communication engine 402 is the part of system 400 that comprises sufficient hardware and logic to setup and dynamically manage the flow of communications between trusted users of the present system. Communication engine 402 may manage communications from disparate means and modes of communications—e.g. text messages, chat, email, voice, video chat and the like.

Multimedia content engine 404 is that part of the system 400 that comprises sufficient hardware and logic to create, store, disseminate and dynamically manage the flow of data in and out of system 400 by and to trusted users of the system. Submodules of engine 404 might advantageously comprise: injest submodule, transcoding submodule, presentation submodule, storage, and delivery submodules.

External ecosystem integration engine 406 may present a set of RESTful API, that allows it to exchange its data with third party systems and using (when applicable) industry standards such as HL7 etc. These API's will allow external systems to send information to the present system, e.g. a medical device or EHR system.

Therapeutics and Research Management Engine 408 is that part of the system 400 that comprises sufficient hardware and logic to create, store, disseminate, and dynamically manage treatment plans and pathways for trusted users on the system. It may be desirable for each trusted user of the system that is actively being treated via system 400 to be tracked by engine 408 and their progress logged and processed. Submodules of engine 408 may advantageously comprise: querio dynamic data capture submodule, therapeutic library, patient education library, and reminders and compliance tracking submodule.

Social networking engine 410 is that part of system 400 that comprises sufficient hardware and logic to dynamically manage the various communications and relationships between trusted users of system 400. It should be appreciated that any known combination of processors, data structures, storage and communication media—including transport of data across networks, intranets, the internet—may be utilized to affect the implementation of the present system, as is known to one skilled in the art.

One aspect of the present system is the ability to transcode, store, deliver and present content of a variety of media types. This would be desirable in any number of applications and context—and one such application is in the field of healthcare where patients may thrive better in a treatment program where use of multiple means of communications and messaging (both synchronous and/or asynchronous) may be applied. For example, a patient may not feel like talking directly to a doctor, or writing a lengthy email about conditions and results; but the patient might be amenable to uploading an audio or video file describing such. So, users and applications can use a multimedia content server/network—such as “anicaport” to affect solutions.

It may also be desirable to create an anicaport in such a way as to build solutions that may have shared content; but it is not desired to transmit the files multiple times. With Anicaport, content files of practically any size can be shared. The content files that are authored in native formats may be uploaded and shared, anicaport may transcodes them to ensure that files will display in Web browser or Mobile device without the need for additional software. In addition, content files may be streamed and transmitted over secure, encrypted protocols and designed to be accessible from anywhere on the globe.

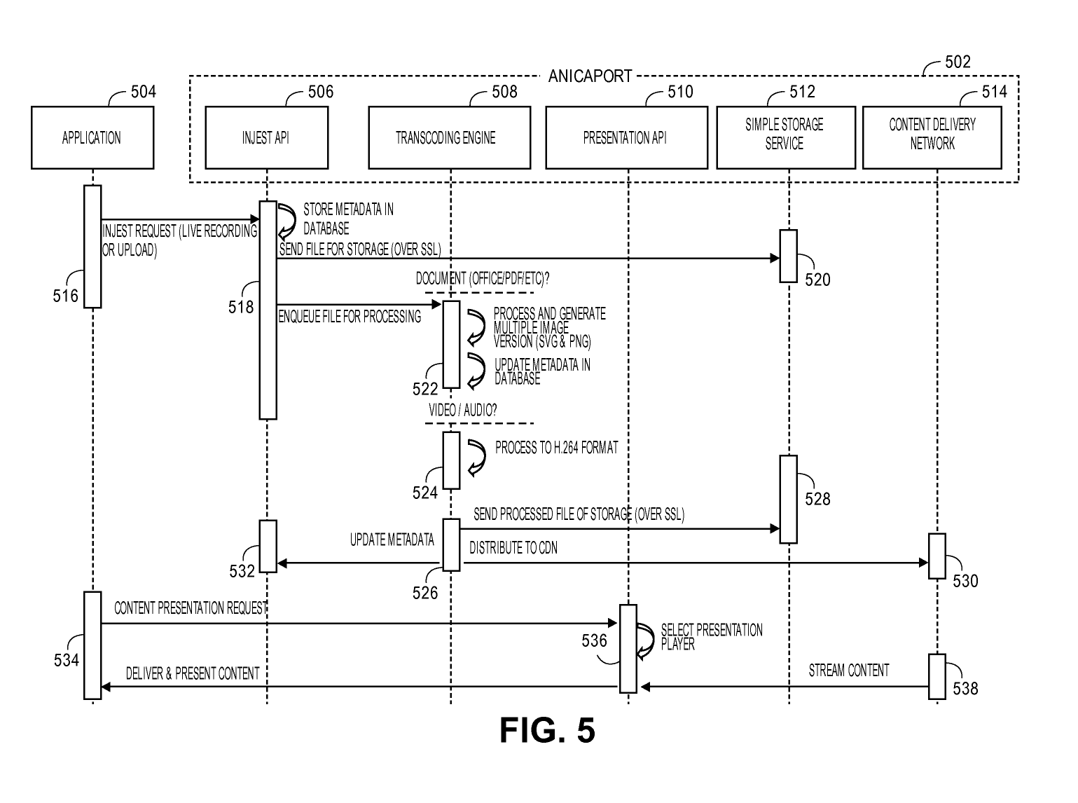

FIG. 5 show one flow chart of the multimedia content engine (“anicaport”) in dynamic operation. Anicaport 502, in this embodiment, comprises injest API 506, transcoding engine 508, presentation API 510, storage 512, and content delivery network 514. Some application (under user control or otherwise) 504 may make an injest request at 516—e.g. a live recording or upload or the like. Injest API 506 may, at 518, store any metadata (if any) in storage or database and send the file associated with the request to storage 520.

This file or data may be queued for further processing at 522 and/or 524, if needed. If the file or data is a form of a document (e.g. office, pdf, etc.), then transcoding engine 508 may process and generate one or more versions, perhaps in different formats, such as image format (e.g. SVG & PNG). Any metadata associated with the transcoding, if any, may be updated in a database or storage. If the file or the data is either an audio or video file, then transcoding engine 508 may process it to a different format—e.g. H.264. Any metadata generated there may also be stored as noted.

At 526, transcoding engine 508 may then send the processed data/file to storage (perhaps over SSL) at 528. In addition, the data/file may be distributed to content delivery network at 530. If there is any update that is needed to earlier saved metadata, it may be accomplished at 532.

Over time, the same or different application 504 may make a request for a presentation of stored content (to which the user or owner of the application has rights to) at 534. Such request may be made to a presentation API 510, which then may select a presentation player at 536 and initiated streaming content at 538 from content delivery network at 538. Presentation API may then oversee such streaming data to application at 534. All of this may be accomplished with the anicaport or other parts of the system checking and enforcing authorizations and permissions—matching users/applications to content.

One embodiment of code that implements an anicaport is shown immediately below. It will be appreciated that many different implementations are possible and are contemplated within the scope of the present invention.

### System Infrastructure

While the architecture of the present system presents one embodiment for the various modules that may be desirable in such a system, the present system itself may be hosted in a myriad of ways, to include leveraging existing infrastructures and the different companies that may provide services and hardware for such hosting and infrastructure.

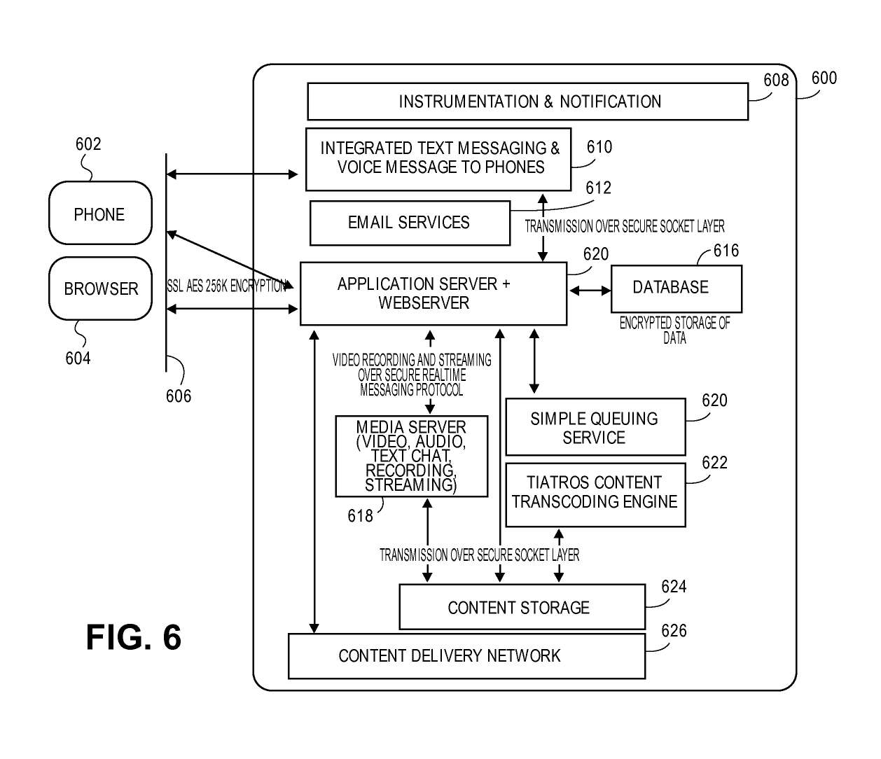

FIG. 6 depicts one embodiment of the present system (600) as it may be hosted over existing infrastructure. Users of the present system may connect by a myriad of communication pathways. For example, users may connect via phone (602), mobile or otherwise, and by a browser 604 through standard interfaces 606. Once connected to the present system 600, the various modules of the present system may be a set of separately hosted modules that are in communication with one another.

The embodiment depicted in FIG. 6 has modules—instrumentation and notification module 608, integrated text and/or voice messaging 610, email service 612, application server and webserver 614, database 616, media server 618, simple queuing service 620, content transcoding engine 622, content storage 624 and content delivery network 626—interconnected in a manner in which each module may be separately hosted, or a set of such modules may be resident on a single site and/or processor.

In one embodiment, the present system may be built on top of best of breed infrastructure available from existing companies—e.g. database hosting services and cloud computing services. It may be desirable that the communication framework of the present system integrates with media servers, SMS gateways and voice capabilities.

In operation, content transcoding engine 622 may convert content files that are uploaded to content storage 624 in any format, e.g., Microsoft Office documents, pdf files, and various image and video formats, preparing them for direct preview and streaming delivery to computing devices, tablet or smartphones (without any downloads). The present system may also advantageously support the sharing of very large image and video content files such as ultrasounds and MRIs. In addition, the present system may also support parallel and separate communication threads among various subsets of a community, ensuring selective and appropriate access to communications, personal health information, and medical reports. The present system may automatically deposit every communication and medical record into a EHR and EMR repository. Notification engine 608 may support therapeutic reminders, workflows and communications.

### Example of Use and Operation

Having described possible architectures and build-out of the present system, it will now be described the uses and operation of an exemplary system, built in accordance with the principles of the present invention.

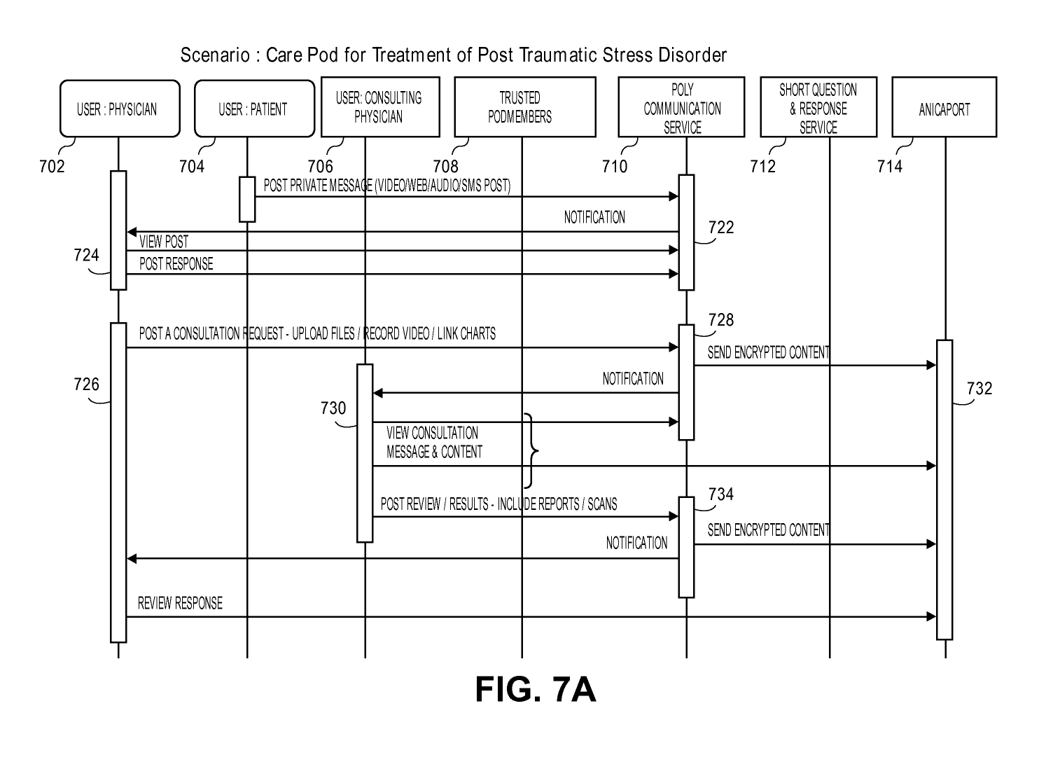

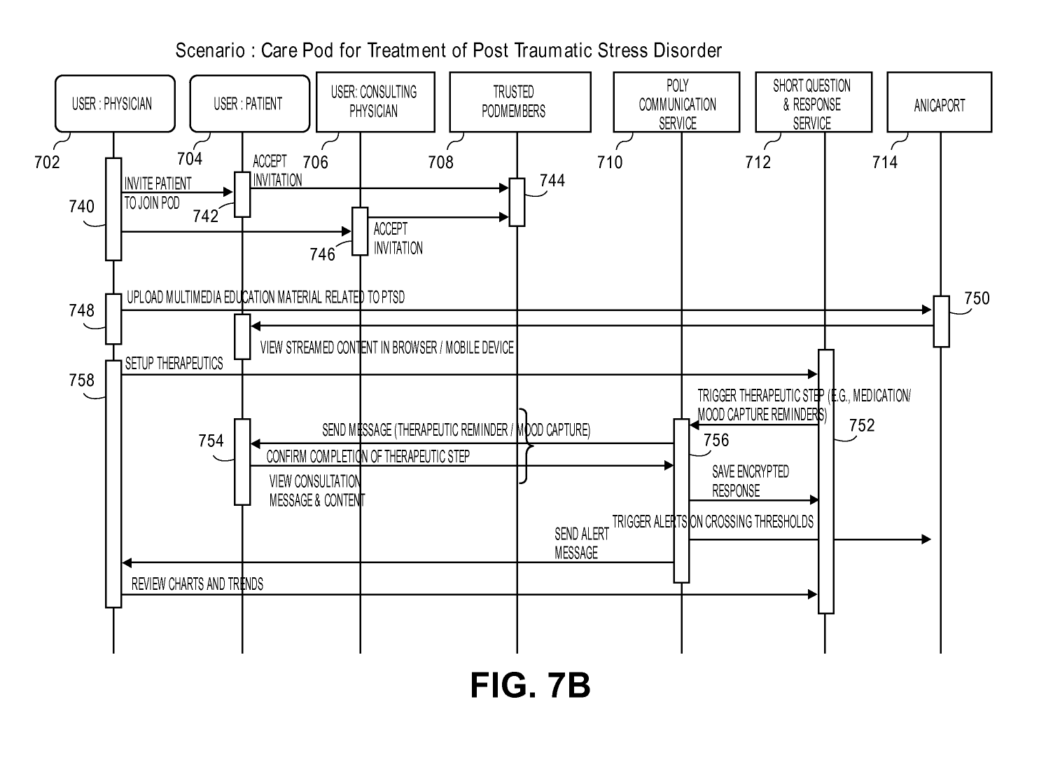

FIGS. 7A and 7B depict the flow of operation of one such embodiment of the present system—i.e. a social pod built and maintained for the management of post-traumatic stress disorder in returning military veterans. It will be appreciated that this embodiment is offered merely for exposition of the present system and does not necessarily limit the scope of invention as claimed below.

In this embodiment, various users may be in communication with other users via and through the present system itself. For example, physicians 702, patient 704, consulting physician 706, other trusted users 708 may be in communication with each other, or various modules of the present system, such as polycommunication service 710, short question and response service 712 and anicaport 714.

In this example, patient 704 may post a private message (at 720, via any known means, e.g. video, web, audio/SMS or the like) meant to be viewed by physician 702. The message may be received by communications service 710 (at 722) and relayed to physician 702 (at 724). Physician 702 may view the post and respond, which is relayed via communication service.

In following-up, physician 702 may post a consultation request at 726 to communication service 710, from which a notification may be sent to consulting physician 706 and a message sent to anicaport 714 at 732. Consulting physician 706 may view the message and content at 730 and then post results of the review back to physician 702 at 734. Anicaport 714 logs all such communications via encrypted content at 732.

In FIG. 7B, physician 702 may invite a new patient 704 and a new consulting physician 706 (at 742 and 746) to join the social pod (as described above) and accept invitations at 744. In addition, physician 702 may decide at 748 to upload certain educational or training materials relating to PTSD to anicaport 750, which then may be viewed by patient 704 as, e.g. streamable content.

Physician 702 may decide to set up a therapeutic regiment for patient 704 at 750. Short question and response service 712 may be employed at 752 to provide reminders and capture any other relevant data (e.g. mood, clinical results, etc) from the patient at 754. If any alert is triggered by the crossing of a threshold (either clinically or via answers or non-compliance noted by the present system), then an alert may be generated and sent to physician at 752, 756 and 750. Lastly, physician 702 may review charts and trends of patient 704 at 752.

### De-Identification

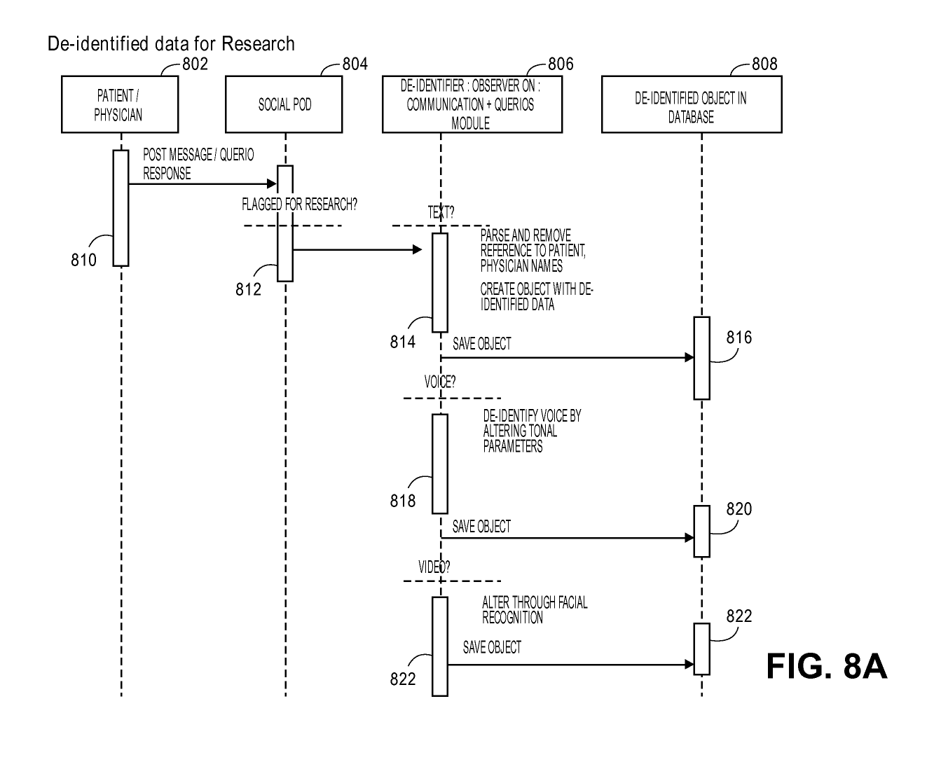

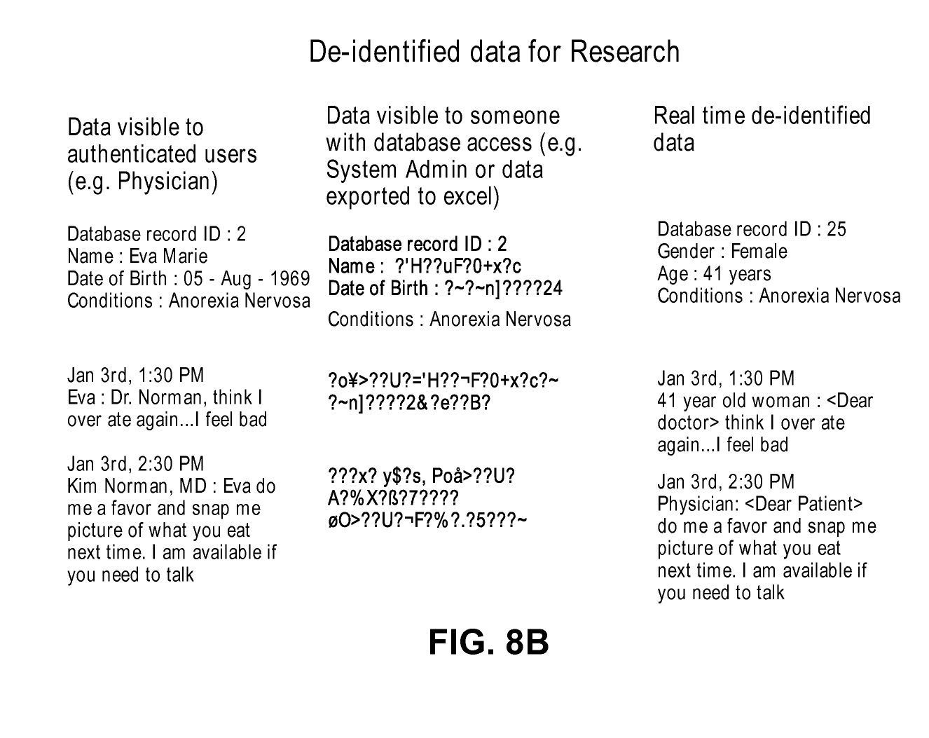

One possible useful feature of a system made in accordance with the principles of the present invention might be the unlinking of patient data from the positive identification of the patient herself. As HIPAA requires that PHI be disseminated in a controlled fashion, FIGS. 8A and 8B depict one embodiment of such a de-identification module.

As noted above, various users of the social pod may be communicating with other users or the system via its interfaces. In this example, physician 802 may be communicating with social pod/system 804. De-identification module 806 may be implemented within the system on top of, or in communication with, query module or communication module or the like. In response, de-identification module 806 may strip out information or data which may be linked to, and help identify, any given patient.

At 810, physician may post a message or a response to the social pod. Such a message, as noted above, may be posted in various forms (e.g. text, voice or video), and it may be desirable that de-identifier module 806 strip out any such identifying data. At 812, such information passed to the social pod 804 may be captured by de-identifier 806. In the case of text at 814, module 806 may parse and remove references to physician and/or patients and create an object without such references, and subsequently be stored at 816.

In the case of voice at 818, module 806 may perform speech recognition to capture information within the message. In addition, module 806 might use voice altering to de-identify the tonal qualities of the individual leaving the message. In the case of video at 822, module 806 may alter pixel data within an image to obscure facial recognition. In addition, module 806 might alter sound and voice data as noted above.

FIG. 8B depicts data and information as may be viewed by either a physician who is authorized to know the identity of the data to whom it is referring—and to others who are not so similarly authorized. The data which is stripped from the data by the present system is depicted in the third column of FIG. 8B.

In one embodiment, the present system may be constructed to capture de-identified data in real-time for research purposes. For example, actual conversations between Physicians/Researchers and Patients (as well as other structured captured data) are typically stored in an encrypted fashion to protect privacy. This however tends to render the data unusable for search and analysis. In these cases, a social pod may be tagged to be “For research”, in which case, the system logs its data in a de-identified form, with the pertinent information but the identifiable elements removed.

### Therapeutics

The present system may also provide a more comprehensive and high engagement support system for better compliance with a therapeutics module. For example, physicians may easily setup a therapeutic action plan and, for each of the components, associate a basket of supporting materials from their online library or record instructions directly through the webcam. The system, will, if setup, send reminders through one or multiple means such as email, voice call or text messages and may require the patient to confirm.

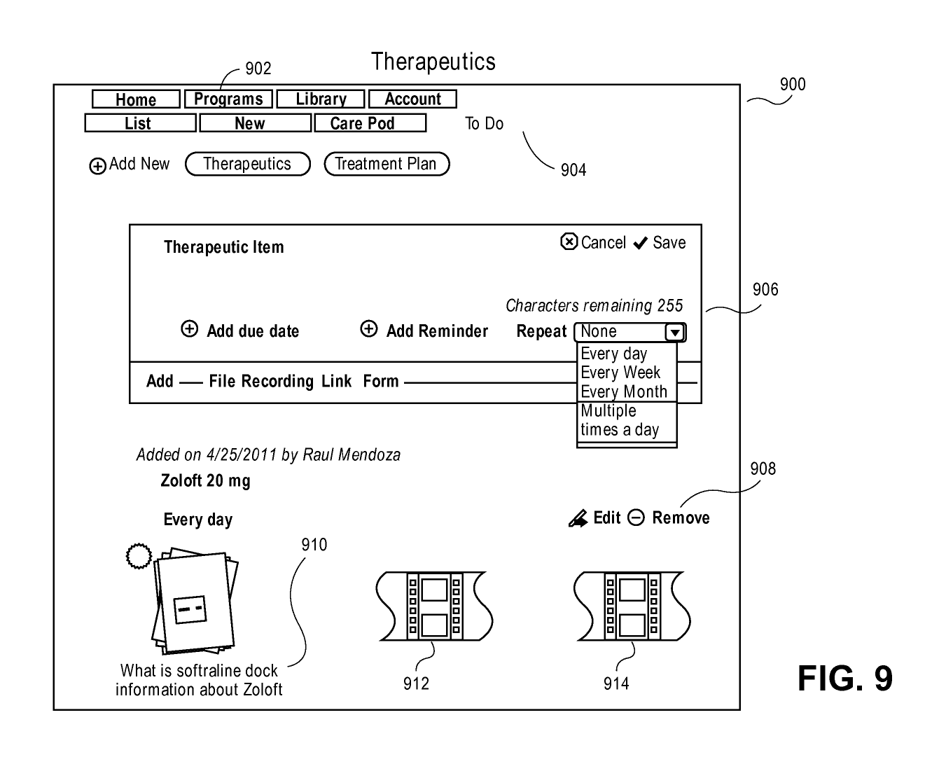

FIG. 9 shows an exemplary screen shot 900 of such a therapeutics interface/module, as may be presented on a web browser or the like. Tabs 902 may be constructed in a user-friendly fashion and, as described above, a To Do tab 904 could be one possible interface to affect a more comprehensive treatment plan for a patient. Possible interfaces might include therapeutic item box 906, where text may be entered by users regarding aspects of the treatment and a set of reminders for treatment may be set in accordance with the treatment plan (e.g. take medications every day, or describe symptoms once a week, take and record vital signs once a month or the like).

In addition, accessible content may be made available through this interface at 908. In this example, the patient has access to a document that describes the medication that she is taking, or the patient may have access to video/audio file 912 that she uploaded to inquire about treatment aspect. Her physician may have responded with a video/audio file 914 in response. Such robust treatment of multimedia content may be delivered as described above.

### Donations and Payments

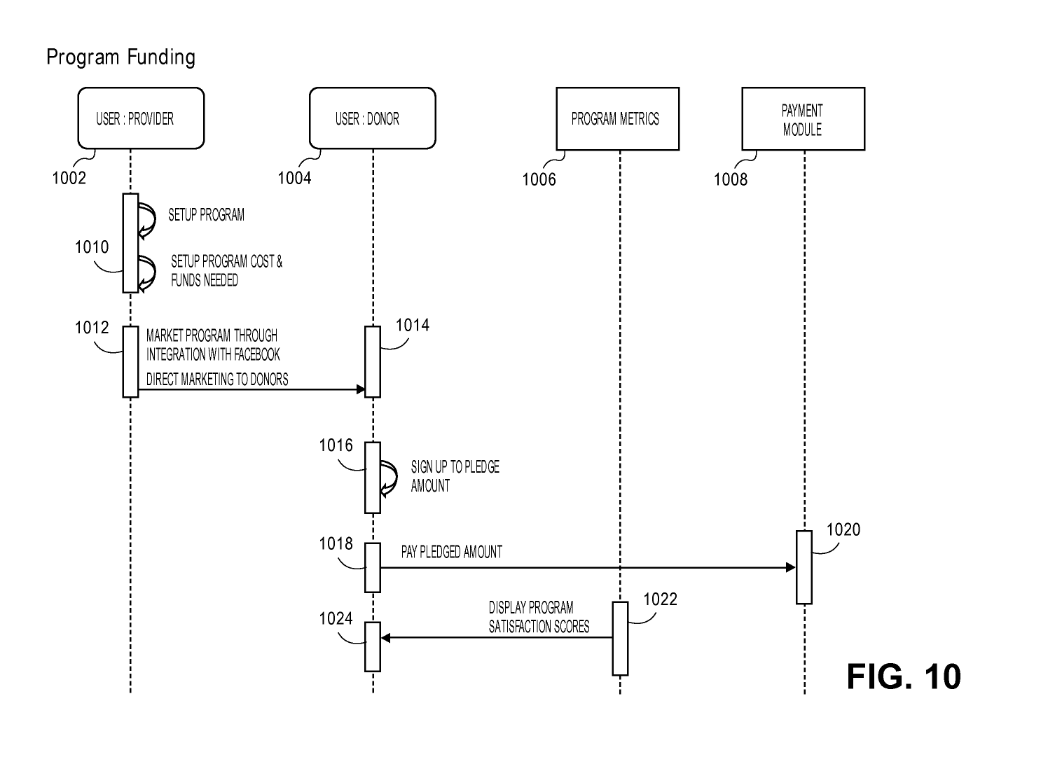

Another aspect of a present system might be a donation and/or payment module that improves the flow of donations and/or payments for programs implemented to address needs of a given social pod. For example, FIG. 10 shows one embodiment of a donation interaction that, in this case, allows providers to raise funds for, e.g., a health outreach program. Donors, in turn, might pledge funds, review outcomes and pay the providers.

Provider 1002 might set up program and outline program cost and funds needed to setup and maintain the program at 1010. Provider 1002 might market such program through any number of channels, e.g. via Facebook or any other social media or outlet at 1012—or market directly to donors. Donors 1004 may receive such marketing at 1014 and pledge some amount at 1016—via the present system. In addition, donors may be made a trusted user and a part of the trusted community, with certain rights and access to materials on the present system. Donor 1004 may make payments at 1018 to payment module 1008 at 1020. To keep the donors informed and engaged, program metrics 1006 may send such metrics—e.g. program satisfaction scores—to donors at 1024, in order for donors to see their donation money at useful work.

### Embodiments of Uploading Medical Data from Patients, Biosensors and Medical Devices

The various embodiments of “Pods” (e.g. “CarePods”, “SocialPods” and the like—the terms may be used interchangeably) described herein (and as further depicted in FIGS. 1-10) create a unique place in the cloud that unifies communication and tools needed to coordinate, manage and provide care to a patient. The CarePod has the ability to provide controlled access to various people involved in the care of a patient within and between Doctor's offices to the various parts of a CarePod, such as the communication tools, the charts and records etc. This capability may allow the parties involved to have a single and common place to go to find the information they need about a patient, even if they are physically distant as well organizationally separated—i.e., they could be part of two different Healthcare providers in two different parts of the world. The charts and records portion of the CarePod, accommodates the storage of records that exists in an electronic form. This provides the opportunity for healthcare providers to upload files that are in electronic form into the CarePod. Several embodiments of the present application herein provide users of CarePods with systems and methods of sharing medical data—generated substantially in real-time (either by patients themselves, by medical devices or by biosensors)—by and between these users.

Embodiments of Real-Time Data Upload to and Sharing Within CarePods

In reference to the discussion below regarding Pods and CarePods (and as further discussed in reference to FIGS. 1-10), it will now be described various embodiments of the uploading and sharing of real-time records into CarePods.

Medical data—either patient-gathered, medical device-gathered or biosensor-gathered—should properly be reviewed by physicians or other health care providers in order to provide proper treatment to the patient. It may be desirable to review this medical data in a near real-time basis, in order to provide timely medical care to the patient.

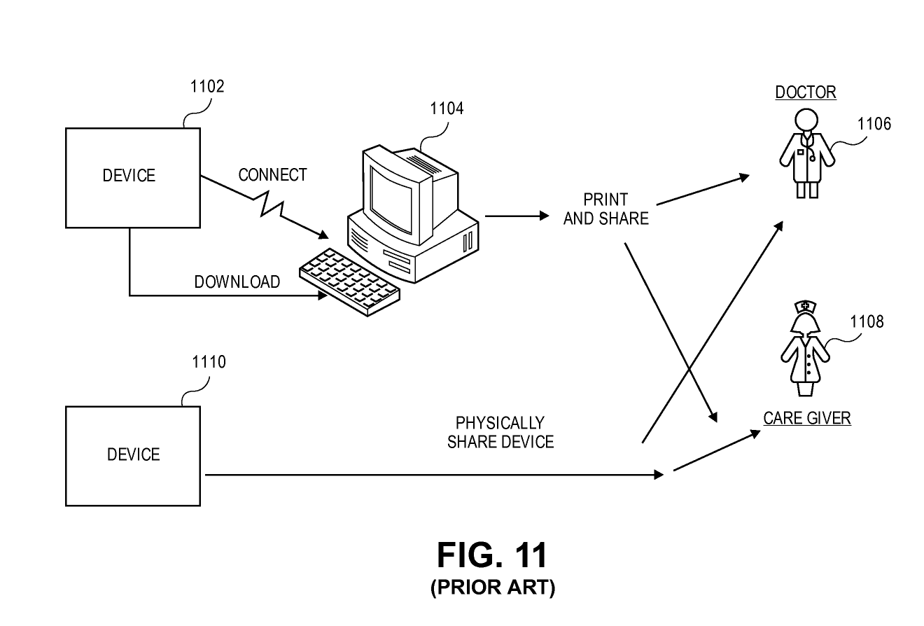

FIG. 11 depicts a plurality of conventional paths by which physicians and other care givers may receive health data (e.g., in substantially real-time) from either patients (tracking their own health data) or from medical devices and/or biosensors (either implantable or external) and possibly gathered from a mobile devices (e.g. iPhone, iPad or the like). In the top path, device 1102 may either create data and the data is manually input into a computer 1104, or may provide the data automatically (via wired, wireless, telemetry or the like). The data is typically then sent to the doctor 1106 and/or care giver 1108, via a physical printout or may be provided electronically to a monitor or the like. In the bottom path, device 1110 may be internal or external to the patient and its data may be shared with the doctor 1106 and/or care giver 1108 substantially in real-time. A record (e.g., on paper or electronically) may be entered thereafter to the patient's record.

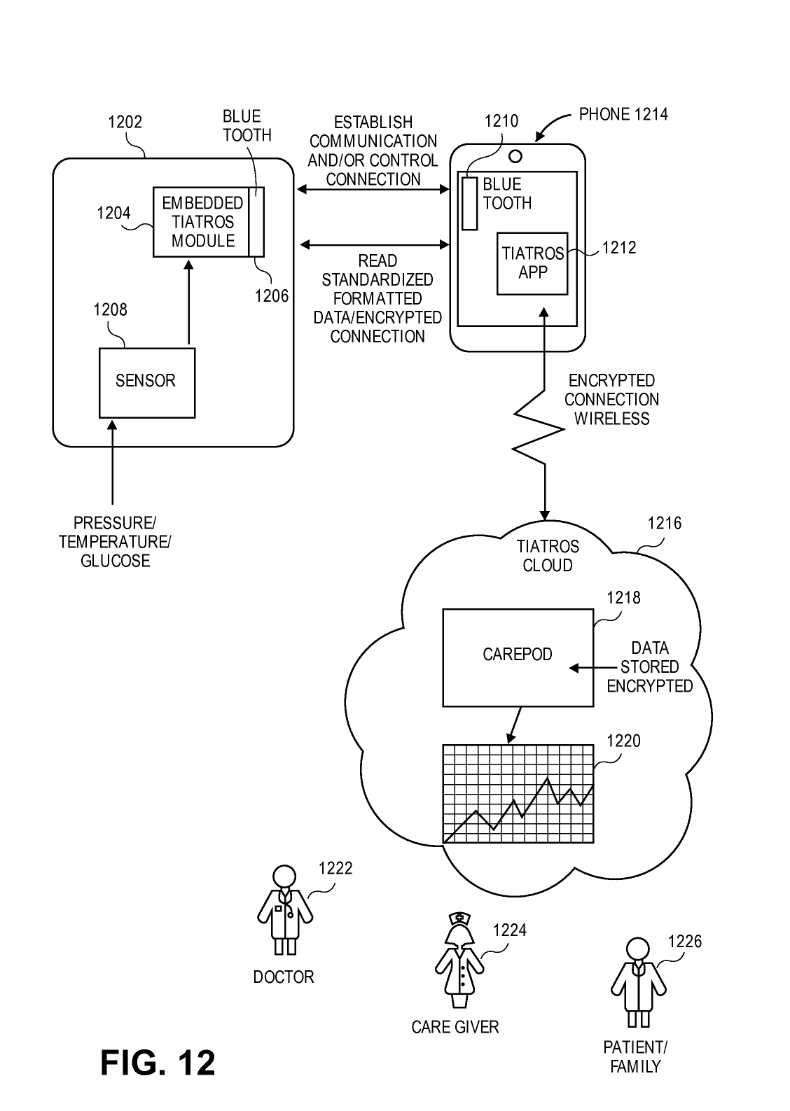

FIG. 12 depicts one embodiment of systems and/or methods for the sharing of real-time data—as might be generated by a medical device, biosensor or mobile application—with other trusted users of a CarePod. Device 1202 may be either internal or external to the patient—and device 1202 may represent a whole host of possible devices, measuring any number of medical and/or physiological conditions (e.g., blood pressure, glucose level, EKG, ECG, implantable sensors or devices or the like). Device 1202 may have one or more sensors 1208. Sensors 1208 may be in command and/or communication connection with communication and/or control module 1204. In one embodiment, module 1204 may be made in accordance with the secure and/or HIPAA compliant systems and methods of the present application.

In one embodiment, sensors 1208 may also comprise active therapy modules as well, such as defibrillator, stimulation or the like, that may be actuated by control signals from module 1204—possibly automatically (e.g., defibrillation) or possibly sent after sensor data 1220 has been reviewed by doctor 1222, care giver 1224, patient family member 1226 and/or other entities within the patient's CarePod 1218.

In one embodiment, module 1204 may comprise, or otherwise be in communication with, a communication module 1206. Communication module 1206 may be wired or wireless communications. As depicted in FIG. 12, communication module 1206 may be compliant to any known or potential wireless standard—e.g., NFC, Bluetooth or the like.

As mentioned, device 1202 may be either internal or external to the patient and device 1202 may be collecting medical data derived from the patient. Such patient information may possibly represent data that should be controlled in a HIPAA compliant manner. In one embodiment, this patient medical data may be communicated ultimately to the patient's CarePod via a communication module 1214. The platform that implements module 1214 may be either a stand-alone communication station (e.g., a desktop, communications port or the like) or a hand-held device (e.g., a phone, smart device or the like).

In one embodiment, module 1214 may also comprises a pairing communication module 1210 (e.g., a wired port, Bluetooth, NFC or the like)—in order to affect communications with device 1202. Through such paired communication modules 1208 and 1210, data may be transmitted from device 1202 to communication module 1214—e.g., by establishing communication and/or command connections and read medical data, possibly in a standardized format and possibly in an encrypted format. This medical data may also be placed in a format that may be employed by patient's CarePod by module 1204. In another embodiment, such data may be formatted for CarePod further downstream in the communications pathway—via, e.g., another module 1212.

From the communication module 1214, medical data may be sent to CarePod 1218 via any suitable communication pathway (e.g., depicted as an encrypted wireless connection in FIG. 12; but may be any other suitable communications pathway that may be capable of transmitting encrypted data, such as wired networks, wireless networks, the Internet or the like).

This data may be communicated to a cloud-based structure 1216, as is known in the art. Cloud structure may comprise various storage and applications to store and possibly modify the medical data. Cloud structure 1216 may also store a plurality of CarePods—which may be constructed for individual patients and/or individuals. The data arriving from device 1202, however, is likely meant to be stored with the CarePod 1218 that is unique to that individual patient.

In other embodiments, it may be possible to utilize data that is de-identified (that is, scrubbed of any identifying indicia of linking the medical data to an individual patient). Such de-identified data may be used by other CarePods or other entities for other purposes—e.g., to supply data for the efficacy of various treatments or therapies or the like.

As mentioned, once the data is uploaded to a patient's CarePod (and possibly in substantially real-time), doctors 1222 and/or care givers 1224 may analyze the data and make treatment decisions in response (e.g. in substantially real-time). In such a case, suitable commands (if meant for device 1202) may be channeled along the same communication pathway as before. Such commands may themselves represent HIPAA compliant data—in which case, the secure and authenticated protocols for communication to and/or from the CarePod would also apply.

### Embodiment to Prevent Accidental Misdirection of Data

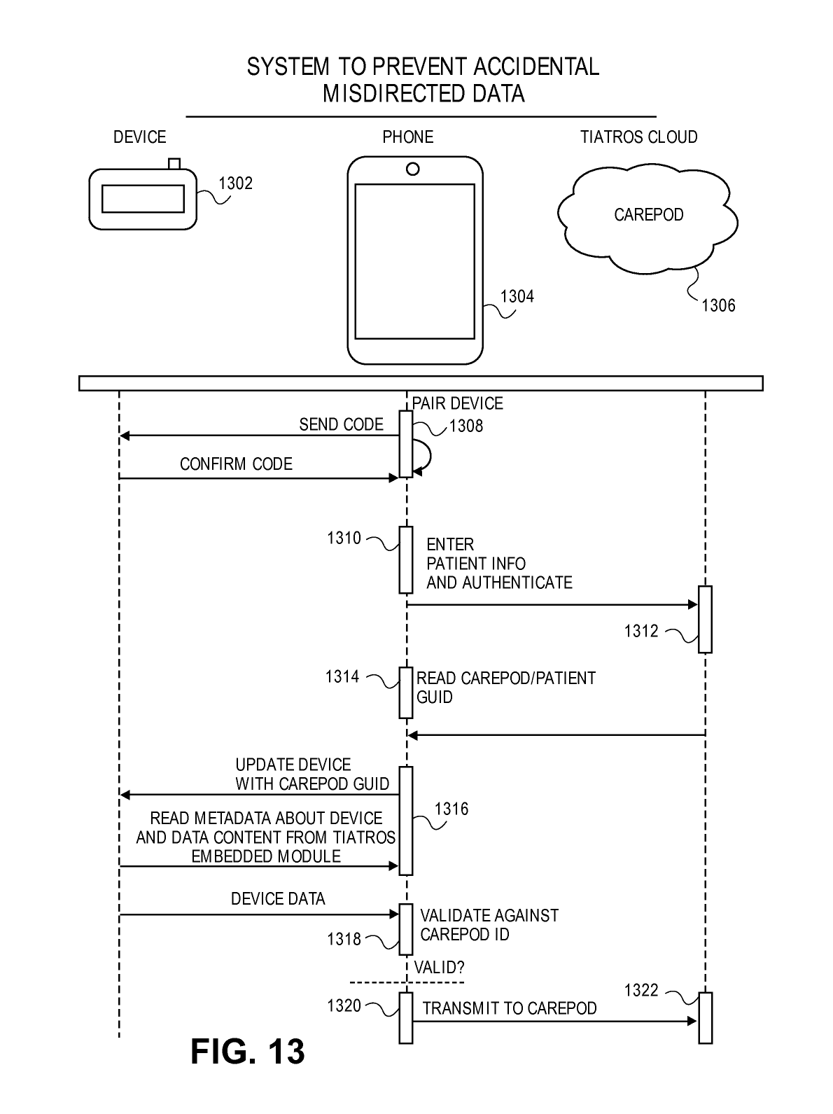

In one embodiment of the present application, care may be desirable in order not to misdirect any sensitive and/or HIPAA compliant patient data to another CarePod that is not associated with individual patient. FIG. 13 depicts one embodiment of a system and/or method to prevent such misdirection.

In one scenario, data may be flowing to and/or from device 1302, via communications module 1304 and ultimately to a cloud structure that comprises the CarePod of the individual patient associated with the data. The cloud structure may also comprise other CarePods for other patients (that are not associated with data). Such cloud structure may be implemented across a number of entities—e.g. practice clinics, hospitals, care centers or the like.

In the present embodiment, communication module 1304 may send (at 1308) a code capable to pairing the communication module 1304 to device 1302. Upon receipt of such a code, device 1302 may confirm receipt of the code. Such code may be used to further secure and authenticate data and communications.

Communications module 1304 may further communicate at 1310 with the cloud structure in order to enter the patient information and authenticate further communications with the CarePod at 1312. CarePod and/or cloud structure may create a Unique Identifier (GUID) that is read by communication module 1304. Such GUID may uniquely identify the patient, the CarePod and/or both—and may be employed to further authenticate communications further that may comprise sensitive patient data.

At 1316, communication module 1304 may update device 1302 with the GUID. In addition, device 1302 may send—and/or communication module 1304 may receive—metadata about the device and data content and/or format from any processing module embedded within device 1302 meant to affect proper formatting for secure and authenticated communications with the CarePod.

At 1318, medical data sensed by device 1302 may be communicated to communication module 1304. The data may have the GUID embedded in any known manner within the data—which may be validated against the GUID that is known to the communication module 1304. If the validation is proper at the communication module 1304, then the data may then be sent to the CarePod that is associated with the individual patient.

It will be appreciated that this GUID check may also occur at other points along the communications pathway from the device to the CarePod—including within the cloud structure itself. An opportunity to perform this check may be made at any point of relaying such information.

Embodiments Comprising Therapeutic Treatment

In many medical situations, patients are admitted to hospital services under substantially emergency conditions. In such cases, key medical data about the patient may often not be accessible during these emergent admissions (e.g., trauma and ICU). In many embodiments of the present application, systems and/or methods may provide a solution linking many modalities seamlessly together to aid in medical provision under one institution, e.g., a Children's Hospital (CH) or the like. Images, lab reports, etc. from different hospitals may all be made available real time under one umbrella.

In this scenario, the patient may be fitted with various devices and sensors and this data (e.g., blood pressure, pulse oximetry, respiration rate) may be uploaded via wired, wireless, telemetry or the like to a HIPAA compliant cloud based server, such as described herein. In one embodiment, doctors may be able to utilize this data (possibly de-identified if desired) for research studies. In another embodiment, set trigger points may be set (either by doctors, care givers or the CarePod on an autonomous or semi-autonomous basis) to elicit a call to 911, the neurosurgeon on call, the nurse, or intensivist on call when a certain threshold value has been reached.

In other embodiments, medical data may be sent from the sensor to a mobile device up to the cloud based server. If for example a sensor was measuring an infant's respiratory rate or EKG, then emergency response services (via 911) as well as the parent's cell phones may be simultaneously triggered to notify the ambulance and parents that concerning readings were being sensed, and that immediate notification and intervention would then be initiated.

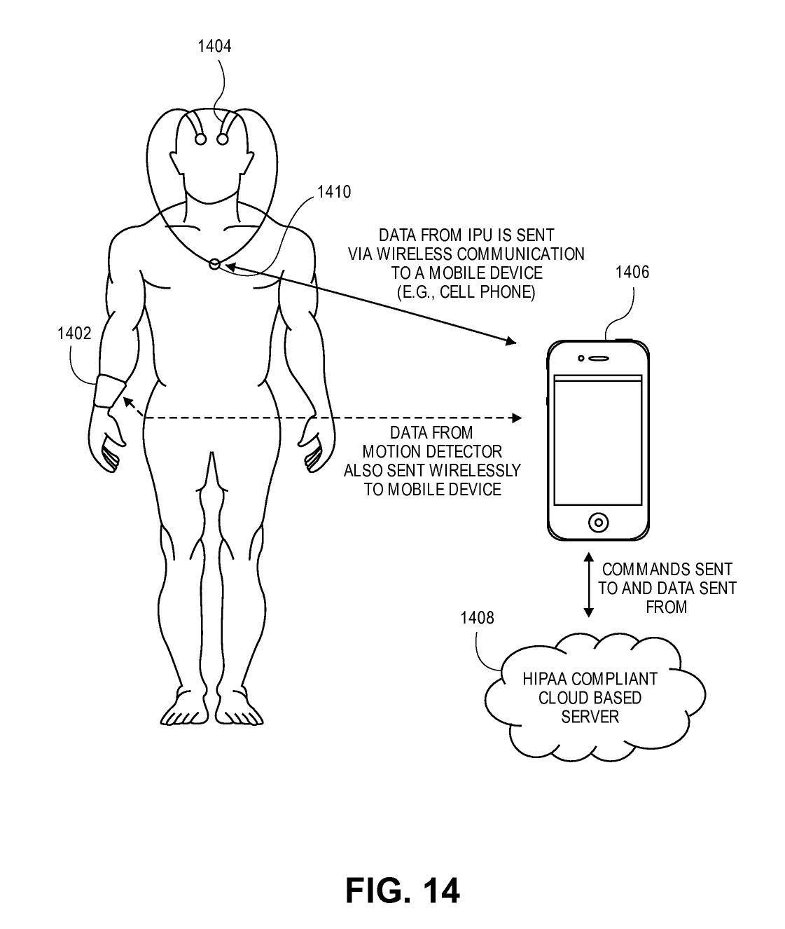

FIG. 14 depicts merely one possible treatment condition that helps to illustrate the therapeutic nature of various embodiments of the present application. In this case, the treatment condition depicted is therapeutic Closed Loop Deep Brain Stimulation (DBS).

Deep brain stimulation (DBS) is a system where electrodes 1404 are placed within the human brain (under an operative procedure) within very specific brain regions. Each electrode has 4 leads, and electrical current is able to pass through a combination of these leads. The actual shape and intensity of the current field is based upon a variety of factors, namely which electrodes are selected to be activated, the amount of the current, the voltage of the current, and the impedance within the circuit. This electrical activity modulates local and general brain function. Primarily, motor function may be improved in Parkinson's patients, but new studies seem to indicate that severe depression may show benefits as well as possible cognition improvements in Alzheimer's. Patients have been noted to show improvements from deep coma as well.

In one application, DBS in children may suffer from motor dyskinesias (abnormal jerky motor movements) as a result of perinatal strokes (eg. Cerebral palsy). Once children are properly selected for surgery (e.g., they have severe motor abnormalities and/or speech disabilities that greatly impact their activities of daily living), they may undergo a procedure for DBS electrode placement. After surgery, they typically would see a neurologist many times to fine tune the DBS system's parameters. Small changes may be made, and the clinician would look for improvements or declines. This is time consuming, and effects are not always apparent after making changes to voltage, current, and other characteristics (e.g., unipolar or bipolar current configuration).

In one embodiment, it may be possible to transmit the data of charge settings back and forth through a radio device 1410 to a mobile phone or other smart device and/or communications module 1406 as discussed above, then this data may be transmitted to a safe, HIPAA compliant cloud-based server in a manner such as disclosed herein.

Apart from the valuable data collection, it may be possible for a neurologist to make changes to the DBS setting remotely, and these changes may be sent via the cloud, to the patient's mobile device, which would then change the device settings on the child's device. This may be time saving and desirable as the care provider of the child may keep a substantially real-time diary and note improvements or worsening of motor function.

In addition, bracelets 1402 may be placed on the patient's arms and/or legs, with a motion sensing device within the bracelet. This would monitor the patient's motor output, and could then be sent to the patient's mobile device and then to a cloud based, HIPAA compliant computer server. It could be employed to correlate motor activity with actual DBS settings. It may thus be possible to monitor change and also see how the patient is doing at all hours, as night monitoring would be automatic and may not be depend on anyone to observe.

During this remote and automatic monitoring, it may be possible to include therapeutic steps that would be able to—with constantly monitoring—change DBS settings (e.g., within carefully set parameters to ensure safety). Such embodiment may allow for closed-loop automatic fine tuning and 24-hour real time adjustments to be made, possibly within pre-set parameters. It is also possible to note that settings may differ while sleeping, after meals, at school; and that DBS changes could be altered accordingly. Circadian rhythms may also be measured. If these diurnal variations are important, than this closed loop monitoring system may be able to pick up these subtleties—and also make the appropriate changes.

This system may enable optimization of the current DBS paradigm at an individual patient perspective. From a global standpoint, it may be possible to study data real time from every child with a DBS, and make appropriate changes and optimizations to hardware, programming paradigms, anatomic localization, etc. all based on studying all data from all patients—leveraged by cloud based, HIPAA compliant platforms.

It will be appreciated that other therapeutic treatments may be envisioned—e.g. other implantable devices could benefit from a closed loop approach. Devices monitoring arrthymias (e.g., Holter monitors) and cardiac pacemakers could also transmit data to the cloud via the above approach. In addition, it is possible to place DBS for epilepsy and create a closed loop system—whereby the wrist or ankle motion detectors may immediately sense abnormal motor discharge (as in a seizure) and then the DBS current may apply a different setting to attempt to dissipate or lessen the abnormal neural discharge, which would cause a generalized seizure to occur.

A detailed description of one or more embodiments of the invention, read along with accompanying figures, that illustrate the principles of the invention has now been given. It is to be appreciated that the invention is described in connection with such embodiments, but the invention is not limited to any embodiment. The scope of the invention is limited only by the claims and the invention encompasses numerous alternatives, modifications and equivalents. Numerous specific details have been set forth in this description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

1. A device for uploading patient medical data to a CarePod, said data associated with an individual for whom said CarePod is further associated, said device comprising:
a sensor for sensing patient medical data;
a communication module for receiving said patient medical data, said communication module further comprising a communication port capable of communicating with said CarePod via a communications pathway; and
wherein further said communication module is capable of receiving a unique identifier from said CarePod to affect communication of said patient medical data to said CarePod associated with said patient.

2. The device as recited in claim 1 wherein said device further comprises a therapeutic module, said therapeutic module is capable to adjusting settings of said device to provide therapeutic treatment to said patient.

3. The device as recited in claim 2 wherein said device capable of providing therapeutic treatment responsive to commands sent from one of a group, said group comprising doctors, caregivers and entities and wherein further said doctors, caregivers and entities are trusted entities of said patient's CarePod.

4. The device as recited in claim 3 wherein said device capable of sending said patient medical data to said CarePod after verification of said unique identifier image.

5. A method for preventing accidental misdirection of sensitive data, the steps of said method comprising:
initializing communications between a device sensing patient medical data and a communications module;
initializing communications between said communications module and the CarePod associated with said patient;
generating a unique identifier, said unique identifier associated with said patient;
sending said unique identifier to said communication module and said device;
sending patient medical data from said device to said communication module;
validating the unique identifier with said CarePod; and
sending patient medical data if said validation is satisified.

6. The method as recited in claim 5 wherein the step of initializing communications with between said device and said communication module further comprises sending a code to authenticate said communications.

7. The method as recited in claim 5 wherein the step of initializing communications between said communications module and said CarePod further comprises sending patient information from said communication module to said CarePod.

8. The method as recited in claim 5 wherein the step of sending said unique identifier to said communication module and said device further comprises sending metadata associated with said device to said communication module.

9. A system for providing therapeutic treatment to a patient from trusted entities with said patient's CarePod, said system comprising
a sensor, said sensor sensing patient medical data for a medical condition for which said patient is capable of receiving treatment;
a therapeutic module, said therapeutic module capable of responding to control commands to set therapeutic treatment;
a communications module, said communications module capable of sending and receiving sensor data and control commands to and from said patient's CarePod respectively

10. The system as recited in claim 9 wherein said communications module is capable of sending authenticated patient medical data to said CarePod in substantially real-time.

11. The system as recited in claim 10 wherein said communications module is capable of receiving authenticated control commands to said therapeutic module from doctors, said doctors being trusted entities for said patient's CarePod and after said doctors having reviewed said patient medical data in substantially real-time.

12. The system as recited in claim 11 wherein said medical condition is one of a group, said group comprising: arrhythmias, DBS, and epilepsy.
